(19) United States
(12) Patent Application Publication
(10) Pub. No.: US 2020/0048415 A1
(43) Pub. Date: Feb. 13, 2020

(54) SOLAR PRODUCTION OF NYLON POLYMERS AND PRECURSORS FOR NYLON POLYMER PRODUCTION

(71) Applicant: ECOLE POLYTECHNIQUE FEDERALE DE LAUSANNE (EPFL), Lausanne (CH)

(72) Inventors: Sophia HAUSSENER, Lausanne (CH); Miguel Antonio MODESTINO, New York, NY (US)

(21) Appl. No.: 16/496,060

(22) PCT Filed: Mar. 20, 2018
(86) PCT No.: PCT/IB2018/051852

§ 371 Date: Sep. 20, 2019

(30) Foreign Application Priority Data
Mar. 20, 2017 (IB) PCT/IB2017/051603

(51) Int. Cl. C08G 69/28; C07C 253/00; F24S 20/20; F24S 23/00; H02S 10/10; H02S 40/22

(52) U.S. Cl. CPC C08G 69/28; C07C 253/00; H02S 40/22; F24S 23/00; H02S 10/10; F24S 20/20

(57) ABSTRACT

The present invention relates to process intensification, and renewable processing routes for polymer production, for example the solar production of Nylon 6,6 and precursors relevant for the Nylon 6,6 production (such as hydrogen, adiponitrile and hexanediamine). The invention deals with the integration of solar energy into the process, specifically aims at petrochemical-free processing, and deals with reformulation of traditional (linear) processes into circular (closed cycle) processing approaches and sustainable processes.

### CROSS-REFERENCE TO RELATED APPLICATIONS

The present application claims priority to PCT Application PCT/IB2017/051603 that was filed on Mar. 20, 2017, the entire contents thereof being herewith incorporated by reference.

### FIELD OF INVENTION

The present invention relates to the production of a polymer of fabric, and more particularly to process intensification, and renewable processing routes for polymer or fabric production, for example the solar production of Nylon 6,6 and precursors relevant for the Nylon 6,6 production (such as hydrogen, adiponitrile and hexanediamine). The invention deals with the integration of solar energy into the process, specifically aims at petrochemical-free processing, and deals with reformulation of traditional (linear) processes into circular (closed cycle) processing approaches and sustainable processes.

The production of polymers and fabrics is an energy-intense process requiring petrochemical inputs. We introduce a novel production approach which replaces the petrochemical inputs by renewable energy (solar energy) and carbon dioxide-neutral carbonaceous resources (biomass or carbon dioxide form the air) increasing the energy efficiency of the process, ensuring cost-competitive and sustainable production of polymers and fabrics. Here, we disclose an invention aiming at such a renewable, petrochemical-free production pathway, specific, but not limited, to the production of Nylon 6,6 and precursors relevant to the production of Nylon 6,6 (such as hydrogen, adiponitrile and hexanediamine). The renewable energy (solar irradiation) is integrated by utilizing for example the radiation in photo-driven electrochemical and solar-driven thermochemical processes and reactors to produce and convert intermediate products (for example, hydrogen, adiponitrile, and hexanediamine) of the complete synthesis route of Nylon 6,6. The approach uses the complete solar spectrum, where mostly the shorter wavelengths are used in the photo-electrochemical route while the longer wavelengths are used in the solar thermochemical routes, after being concentrated.

### BACKGROUND

The production of fabrics is an energy-intense process requiring petrochemical inputs. Novel production approaches replacing petrochemical inputs by carbon-dioxide from the air captured by plant photosynthesis can mitigate problems arising from the decline of fossil resources, volatility in fuel prices, and environmental issues. They can furthermore benefit the environment by: i) omitting the use of any fossil, non-renewable resource and the accompanying pollution and environmental issues related to its extraction, transport, and processing, ii) improving the efficiency of the process and therefore reducing the need for energy input, and iii) replacing the energy input by a renewable and sustainable source.

### SUMMARY

The present invention addresses the above-mentioned inconveniences and problems and provides a Fabric or polymer production process according to claim 1, and a fabric or polymer production system according to claim 11.

Further advantages features are present in the dependent claims.

In the process and system of the present disclosure, the efficiency increases, decreasing the production cost and CO₂ emissions, and transforming the current, linear production approach into a sustainable, cyclic process. Specifically, an approach is disclosed for the solar production of petrochemical-free Nylon 6,6 and relevant precursors (FIG. 1). The process uses for example one part of the solar energy in a photovoltaic array, which drives for example the electro-chemical reduction of aqueous streams of acrylonitrile (ACN) to adiponitrile (ADN) and hydrogen. This process can be closely integrated (i.e. a photo-electrochemical approach) or composed of separated PV and electrochemical units. The second part of the solar energy is used in three different thermochemical steps conducted in high-temperature solar reactors. The first converts ADN and hydrogen to hexanediamine (HDA), which is the main constituent of Nylon 6,6. As ACN can be obtained from biomass, the process will effectively fixate environmental CO₂ into the final fabric, transforming the Nylon process from a linear to a circular model. The second thermochemical process converts ADN to adipic acid (ADA). The third thermochemical process concerns the polymerization of adipic acid ADA with 1,6-hexyldiamine HDA in order to produce, for example, Nylon 6,6.

This disclosure promotes a process with increased efficiency and which doesn't use any fossil fuel or fossil energy resources. This process will not emit carbon-dioxide. In its final form, the process will use as inputs only biomass-derived ACN, water, and the sun. Plants capture and sequester carbon-dioxide from the air in their photosynthetic production of biomass. By implementing biomass feedstocks in this process, the carbon-dioxide is recycled and used in the production of green polymers, fabrics and textiles, which effectively are made of carbon-dioxide sequesters from the environment, reducing the carbon-dioxide in the atmosphere. Overall, our process is a sustainable, cyclic process with reduced energy inputs and reduced emission and pollution outputs.

The above and other objects, features and advantages of the present invention and the manner of realizing them will become more apparent, and the invention itself will best be understood from a study of the following description with reference to the attached drawing showing some preferred embodiment of the invention.

### BRIEF DESCRIPTION OF THE DRAWING

For purposes of clarity, not every component may be labeled in the drawing. The drawing is not necessarily drawn to scale, with emphasis instead being placed on illustrating various aspects of the techniques and devices described herein.

The above object, features and other advantages of the present disclosure will be best understood from the following detailed description in conjunction with the accompanying drawing, in which:

FIG. 1 presents a schematic diagram of the process and system for the production of Nylon 6,6 using solar energy inputs.

301 corresponds to the visible and ultraviolet part of the solar spectrum (or part of the visible and ultraviolet part of the solar spectrum), while 302 corresponds to the infrared IR component (or part of the infrared IR component of the solar spectrum). 303 is the transmitted or reflected IR component of the spectrum that is then concentrated into the irradiation streams 304. 101 is a photovoltaic component that selectively absorbs the visible and ultraviolet part of the spectrum, and reflects or transmits the IR portion of it. 102 is a solar concentrator. 201 is an electrochemical reactor for the production of ADN.

202, 203 and 204 are solar-thermal reactors for respectively the hydrogenation of ADN, the hydrolysis of ADN and the step-polymerization of HDA with ADA into Nylon 6,6.

The chemical species 401, 402, 403, 404, 405, 406, 407, 408 and 409 correspond respectively to water, oxygen, protons, acrylonitrile, ADN, hydrogen, HDA, ADA, and Nylon 6,6.

### DETAILED DESCRIPTION

This present disclosure concerns a process and system for the production of a fabric or polymer, for example, for the production of Nylon 6,6 and precursors relevant for the Nylon 6,6 production using solar irradiation as an energy source.

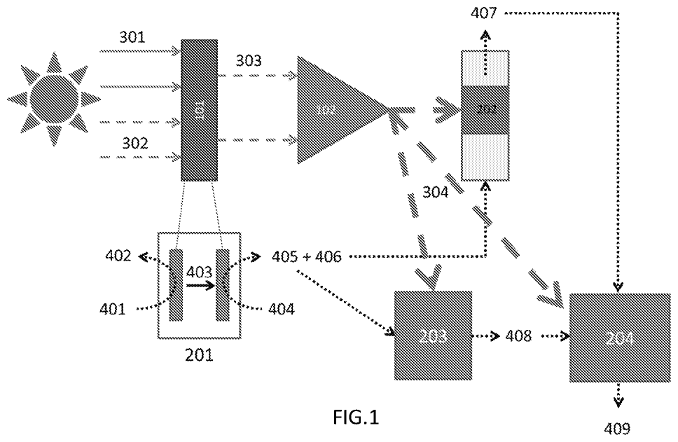

FIG. 1 presents a schematic diagram describing the system and process and the chemical transformations that take place in each of the units of the system and process.

The system may include, for example, a photovoltaic module 101 and an electrochemical reactor 201. The system may alternatively or additionally include one or a plurality of solar-thermal (thermochemical) reactors 202, 203, 204 as well as a solar concentrator 102.

The solar concentrator 102 is configured to receive infrared IR radiation from a source (for example, the photovoltaic module 101) and distribute the IR radiation to the solar-thermal (thermochemical) reactors 202, 203, 204.

The photovoltaic component or photovoltaic module 101 can, for example, comprise materials with band gaps tailored to a required transmittance for visible and/or IR radiation to be used in the solar concentrator 102 for distribution to reactor elements of the systems. The PV component is or comprises, for example but not limited to, polycrystalline silicon PV cells. Dichroic filters can, for example, alternatively be used to selectively transmit and/or reflect light onto the PV cell and the solar concentrator.

The solar concentrator 102 is configured to concentrate the solar light and/or the transmitted or received IR light using or with line focusing (for example, parabolic through or linear Fresnel systems) or point focusing (for example, solar dish or solar tower systems) mirror systems or lens systems.

The units involved in the process may for example include or consist of a photovoltaic module 101, a solar concentrator 102, an electrochemical reactor 201 for the production of for example adiponitrile (ADN) and H₂, and, for example, three solar-thermal reactors 202, 203 204 for the hydrogenation of for example ADN, the hydrolysis of ADN, and the solid-state step polymerization of HDA and ADA into Nylon 6,6, respectively.

The photovoltaic module 101 is configured to absorb the visible and ultraviolet part 301 of the solar spectrum and to provide the voltage and current required for the electrochemical reactor 201. In the electrochemical reactor 201, water 401 is provided thereto and reacts at the anode of the electrochemical reactor 201 to produce oxygen 402 and protons 403. Protons 403 migrate from the anode to the cathode the electrochemical reactor 201 and combine with acrylonitrile ACN to produce adiponitrile ADN. A fraction of the protons 403 is reduced by the electrochemical reactor 201 to produce hydrogen 406.

The electrochemical reactor 201 may be composed of, but not limited to, two plate electrodes (an anode and a cathode). The anode can be made of, but not limited to, Carbon, Nickel, Iron or Iridium oxide. The cathode can be made of, but not limited to, Cadmium, Lead or Platinum. The gap between the plate electrodes can be filled with an electrolyte composed of for example a combination of, but not limited to, ACN, water, phosphate salts, quaternary ammonium salts, and Ethylenediaminetetraacetate salts. The electrolyte may be incorporated in static or flowing conditions inside the cell of the electrochemical reactor 201.

The mixture of ADN 405 and hydrogen 406, which may come from the electrochemical reactor 201 and/or from other sources, is then provided or sent to a solar-thermal (thermochemical) reactor 202 configured for the hydrogenation of ADN 405 into 1,6-hexyldiamine (HDA).

The thermochemical reactor 202 may be, for example, a batch type or flow reactor, for example, composed of multiple tubes (or other channel geometries) directly or indirectly irradiated by the concentrated solar irradiation 304. The tubes can be nano, meso or macro-scaled, or made of combinations thereof, and can be made of opaque (for example, graphite or steel) or transparent materials (for example, quartz). The tubes and possibly a porous substrate within the tube are partially covered with, but not limited to, iron or cobalt-catalysts. The concentrated IR irradiation may come, but is not restricted to, from the portion 303 of the spectra that was transmitted by or reflected from the photovoltaic component 101.

Part of the ADN produced by the electrochemical reactor 201 is sent or provided to a solar-thermal (thermochemical) reactor 203 where it hydrogenated and subsequently hydrolysed to adipic acid 408 (ADA), the solar-thermal (thermochemical) reactor 203 being configured for this purpose.

The reactor used can follow or be configured according to, but is not limited to, the design described in reference 1, the entire contents thereof being herewith incorporated by reference.

The concentrated IR irradiation may come, but is not restricted to, from the portion 303 of the spectra that was transmitted by or reflected from the photovoltaic component 101.

In the last part of the process, ADA 408 reacts with HDA 407 in a solar-thermal polymerization reactor 204 to produce Nylon 6,6.

The reactors for this reaction can follow or be configured according to, but are not limited to, the designs used in commercial nylon production, described in reference 2, the entire contents thereof being herewith incorporated by reference. All of the solar-thermal reactors 202, 203, 204 may use, but are not restricted to, concentrated infrared radiation coming or provided from the solar concentrator.

The system of the present disclosure thus concerns a fabric or polymer production system. The system may include at least one biomass-derived material or biomass-derived material source (and/or biomass-derived material feeder), at least one reactor 201,202,203,204 for transforming the biomass-derived material, and a solar energy converter apparatus 101, 102 configured to supply thermal energy or electrical energy to the at least one reactor 201,202,203,204.

The solar energy converter apparatus 101, 102 is configured to convert a first part of the solar energy into electrical energy to operate the reactor 201, and configured to concentrate a second part of the solar energy on one or a plurality of the reactors 202,203,204 to operate these reactors.

The reactor 201 is configured to transform the biomass-derived material into a first product or products, and the second reactors 202,203 are configured to transform the first product or products into a second product or products.

As shown in FIG. 1, the system can include the electrochemical reactor 201, thermochemical reactor 202, thermochemical reactor 203 and a thermochemical reactor 204.

The solar energy converter apparatus 101 is configured to convert a first part of the solar energy into electrical energy to operate the electrochemical reactor 201, and the solar energy converter apparatus 102 configured to concentrate a second part of the solar energy on the thermochemical reactors 202,203,204 to operate these reactors.

The electrochemical reactor 201 is configured to transform the biomass-derived material (for example acrylonitrile (ACN)), into the first product or products (for example, adiponitrile (ADN)) and the system is configured to provide these first product or products to the thermochemical reactor 202 and the thermochemical reactor 203.

The thermochemical reactor 202 is configured to transform the first product or products into a second product or products (for example, 1,6-hexyldiamine (HDA)) and the thermochemical reactor 203 is configured to transform the first product or products into a third product or products (for example, adipic acid (ADA)).

The system is also configured to provide the second and third product or products to the thermochemical reactor 204 and the thermochemical reactor 204 is configured to transform the second and third products or products into a fourth product (Nylon 6,6 or a Nylon polymer).

The solar energy converter apparatus may include a photovoltaic module 101 to provide to electrical energy and a solar concentrator 102 to provide thermal energy.

As previously mentioned, the thermochemical reactor 202 can be configured for example to hydrogenate adiponitrile (ADN) in order to produce 1,6-hexyldiamine (HDA), the thermochemical reactor 203 can be configured for the hydrolysis of adiponitrile in order to produce adipic acid (ADA), and the thermochemical reactor 204 can be configured for step-polymerization of adipic acid (ADA) with 1,6-hexyldiamine (HDA) in order to produce Nylon 6,6 or a Nylon polymer.

While the present disclosure discloses the production of the particular Nylon polymer that is Nylon 6,6, it should be noted that other fabrics or Nylon polymers may also be produced using the process and system of the present disclosure which is not limited to solely the production of Nylon 6,6 for example but not limited to, Nylon 6,10 or Nylon 6/66 or Nylon 1,6 can also be produced using the process and system of the present disclosure.

While the invention has been disclosed with reference to certain preferred embodiments, numerous modifications, alterations, and changes to the described embodiments, and equivalents thereof, are possible without departing from the sphere and scope of the invention. In particular, the features of any one embodiment may be combined with the features of any other embodiment. Accordingly, it is intended that the invention not be limited to the described embodiments, and be given the broadest reasonable interpretation in accordance with the language of the appended claims.

### REFERENCES

[1] R. di Felice, A. Bottino, G. Capannelli, A. Comite, and T. di Felice, Kinetics of Adiponitrile Hydrogenation over Rhodium-Alumina Catalysts, International Journal of Chemical Reaction Engineering, vol. 3, 2005
[2] Tzoganakis, C., Reactive extrusion of polymers: A review, Advances in Polymer Technology, 1989, 9, 321-330.

1. Fabric or polymer production process comprising the steps of:
providing solar energy as input energy to the production process; and
using the provided solar energy and/or energy converted from the provided solar energy to supply energy to at least one electrochemical system and/or at least one solar-thermochemical reactor to produce the fabric or polymer, or to produce intermediate products for the production of the fabric or polymer.

2. The process of claim 1, wherein the solar spectrum of the provided solar energy is absorbed by a photovoltaic array and the electricity generated is used in the at least one electrochemical system for the production of adiponitrile.

3. The process of claim 2, wherein where the solar spectrum is absorbed by the photovoltaic array selectively absorbing ultraviolet and visible irradiation, and infrared radiation of the solar spectrum is reflected or transmitted by the photovoltaic array, and the electricity generated by the photovoltaic array is used in the electrochemical system for the production of adiponitrile (ADN).

4. The process of claim 1, wherein the photovoltaic array selectively absorbs ultraviolet and visible irradiation and reflects or transmits infrared radiation, and the electricity generated is used in an electrochemical system for the production of adiponitrile.

5. The process of claim 1, wherein solar irradiation is concentrated in a solar concentrator and fed to at least one or a plurality of solar-thermochemical reactors.

6. The process of claim 4, wherein the part of the solar spectrum that was transmitted or reflected from a photovoltaic module is concentrated in a solar-concentrator and delivered to at least one or a plurality of solar-thermochemical reactors.

7. The process of claim 1, wherein a solar-thermochemical reactor is used for the hydrogenation of adiponitrile (ADN) in order to produce 1,6-hexyldiamine.

8. The process of claim 1, where a solar-thermochemical reactor is used for the hydrolysis of adiponitrile in order to produce adipic acid.

9. The process of claim 1, where a solar-thermochemical reactor is used for the step-polymerization of adipic acid with 1,6-hexyldiamine in order to produce Nylon 6,6.

10. The process of claim 1, wherein the fabric or polymer is Nylon 6,6 or Nylon 6,10 or Nylon 6/66 or Nylon 1,6.

11. Fabric or polymer production system including:
at least one biomass-derived material source;
at least one reactor for transforming the biomass-derived material; and
a solar energy converter apparatus configured to supply thermal energy or electrical energy to the at least one reactor.

12. System according to claim 11, wherein the solar energy converter apparatus is configured to convert a first part of the solar energy into electrical energy to operate a first reactor, and configured to concentrate a second part of the solar energy on a second reactor to operate the second reactor.

13. System according to claim 12, wherein the first reactor is configured to transform the biomass-derived material into a first product or products, and the second reactor is configured to transform the first product or products into a second product or products.

14. System according to claim 11, wherein the system includes an electrochemical reactor, a first thermochemical reactor, a second thermochemical reactor and a third thermochemical reactor.

15. System according to claim 14, wherein the solar energy converter apparatus is configured to convert a first part of the solar energy into electrical energy to operate the electrochemical reactor, and configured to concentrate a second part of the solar energy on the first, second and third thermochemical reactors to operate the said reactors.

16. System according to claim 14, wherein the electrochemical reactor is configured to transform the biomass-derived material into the first product or products and the system is configured to provide the first product or products to the first thermochemical reactor and the second thermochemical reactor.

17. System according to claim 15, wherein the first thermochemical reactor is configured to transform the first product or products into a second product or products and the second thermochemical reactor is configured to transform the first product or products into a third product or products.

18. System according to claim 15, wherein the system is configured to provide the second and third product or products to the third thermochemical reactor and the third thermochemical reactor is configured to transform the second and third products or products into a fourth product.

19. System according to claim 1, wherein the solar energy converter apparatus includes a photovoltaic module to provide to electrical energy and a solar concentrator to provide thermal energy.

20. System according to claim 1, wherein the biomass-derived material includes acrylonitrile, the first product or products include adiponitrile, the second product or products include 1,6-hexyldiamine, the third product or products include adipic acid and the fourth product includes a Nylon polymer, or Nylon 6,6 or Nylon 6,10 or Nylon 6/66 or Nylon 1,6.

21. System according to claim 1, wherein the first thermochemical reactor is configured to hydrogenate adiponitrile in order to produce 1,6-hexyldiamine.

22. System according to claim 1, wherein the second thermochemical reactor is configured for the hydrolysis of adiponitrile in order to produce adipic acid.

23. System according to claim 1, wherein the third thermochemical reactor is configured for step-polymerization of adipic acid with 1,6-hexyldiamine in order to produce a Nylon polymer, or Nylon 6,6 or Nylon 6,10 or Nylon 6/66, or Nylon 1,6.
